(19) United States
(12) Patent Application Publication
(10) Pub. No.: US 2015/0359885 A1
(43) Pub. Date: Dec. 17, 2015

(54) THERMAL THERAPEUTIC REAGENT

(71) Applicant: Jen-Jie Chieh, Taipei City (TW)

(72) Inventors: Herng-Er Horng, New Taipei City (TW); Hong-Chang Yang, New Taipei City (TW); Chin-Yih Hong, Changhua County (TW); Chau-Chung Wu, Taipei City (TW); Kai-Wen Huang, Taipei City (TW); Jen-Jie Chieh, Taipei City (TW); Shu-Hsien Liao, New Taipei City (TW)

(21) Appl. No.: 14/303,588

(22) Filed: Jun. 12, 2014

(51) Int. Cl. A61K 41/00; A61K 33/26; A61K 9/51

(52) U.S. Cl. CPC A61K 41/0052; A61K 9/5161; A61K 33/26

(57) ABSTRACT

A thermal therapeutic reagent is provided. The thermal therapeutic reagent comprises a plurality of magnetic nanoparticles, a plurality of surfactants coating on the magnetic nanoparticles respectively, and a polar magnetic fluid for delivering the magnetic nanoparticles to a target site. Wherein, the magnetic nanoparticles are capable of being activated under a magnetic field applied at the target site. Another thermal therapeutic reagent comprising a non-polar magnetic fluid is also provided.

### BACKGROUND OF THE INVENTION

1. Field of the Invention

The present invention generally relates to treatments of tumor tissue, in particular, to thermal therapeutic reagents for treating cancerous tissue.

2. Description of Related Art

Local ablation of the tumor using minimally invasive techniques is a recognized form of treatment if the tumor is relatively small. Current local ablation modalities are typically easily performed, safe and repeatable procedures, and may include percutaneous ethanol injection (PEI), percutaneous acetic acid injection (PAI), radiofrequency ablation (RFA) and microwave ablation (MWA) therapy.

The mechanisms of PEI or PAI are based on the dehydration, and intracellular protein damage on the tumor cells. However, PEI or PAI are often unable to produce homogeneous distribution of ethanol within a tumor. Currently, RFA or MWA is generally used for the treatment and/or palliation of solid tumors in patients who are nonsurgical candidate. The mechanisms of RFA or MWA are based on the generation of heat between the tissue and electric current or microwave emitted by an RF or microwave electrode into the tumor, respectively, wherein the heat causes coagulation, followed by cellular death as soon as the temperature in the target area exceeds about 60° C. Nevertheless, the main limitations of RFA and MWA may include limitations of ablation volume, technically infeasible in some tumors due to conspicuity and dangerous location, and the heat-sink effect.

Transcatheter arterial embolization (TAE) or chemoembolization (TACE) are other therapies for patients with noninvasive tumors in intermediate-stage disease. TAE refers to the embolization of the artery without using any chemotherapeutic agents. When TAE is combined with prior injection into the artery of chemotherapeutic agents, the procedure is known as TACE. These techniques are performed through puncturing of the common femoral artery or a selected branch of the artery and injecting embolic agents alone or linked to a chemotherapeutic drug. The embolic agents will close the artery which provides blood to the liver volume in which there is the tumor, and thus occlusion will cause an ischemic necrosis of the nodule and of the healthy surrounding tissue. However, the limitations of TAE or TACE are represented by the difficulties in obtaining a complete necrosis of the lesion treated, which made patients require repeated TAE or TACE treatments. In particular, chemotherapeutic drugs are not easily controlled to be selective to tumor cells, thus residue tumor proliferation, tumor recurrence and metastasis after TACE may influence long-term outcome, producing very severe side-effects.

### SUMMARY OF THE INVENTION

Accordingly, the invention provides thermal therapeutic reagents in which magnetic nanoparticles are delivered to tumor tissues and activated under magnetic field to generate heat, and thereby enhance higher inductive heating efficacy for locoregional treatments.

The invention provides a thermal therapeutic reagent comprising a plurality of magnetic nanoparticles, a plurality of surfactants coating on the magnetic nanoparticles respectively, and a polar magnetic fluid for delivering the magnetic nanoparticles to a target site. Herein, the magnetic nanoparticles are capable of being activated under a magnetic field applied at the target site.

The invention also provides a thermal therapeutic reagent comprising a plurality of magnetic nanoparticles, a plurality of surfactants coating on the magnetic nanoparticles respectively, and a non-polar magnetic fluid for delivering the magnetic nanoparticles to a target site. Herein, the magnetic nanoparticles are capable of being activated under a magnetic field applied at the target site.

In view of foregoing, the magnetic nanoparticles of the provided thermal therapeutic reagents may enhance heat transfer of the heat generated around tumor tissues during locoregional treatments. As such, the healing efficacy to cause the death of tumor cells will be improved.

In order to make the aforementioned and other features and advantages of the present application more comprehensible, several embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram illustrating a thermal therapeutic reagent.

FIG. 2 is a schematic diagram illustrating a magnetic field generator for generating an adjustable magnetic field.

FIG. 3 is a schematic diagram illustrating a thermal therapeutic reagent binding on a tumor cell.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

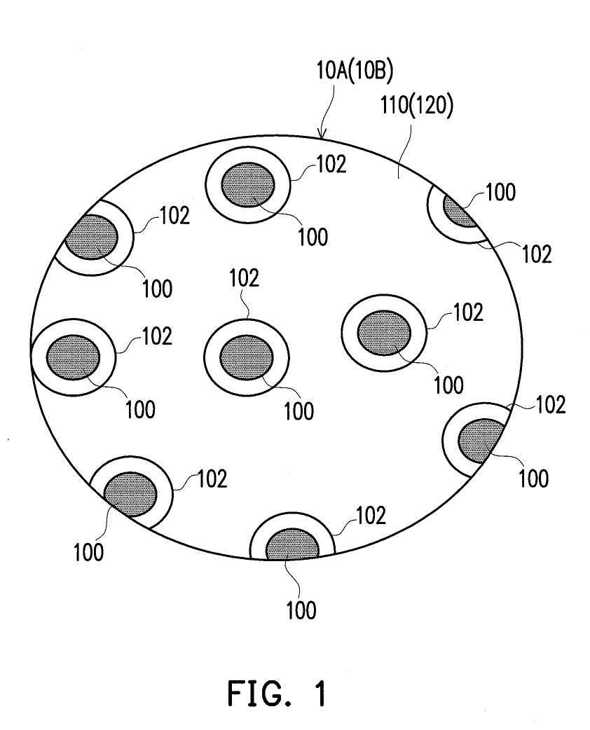

FIG. 1 is a schematic diagram illustrating a thermal therapeutic reagent. Referring to FIG. 1, the thermal therapeutic reagent 10A of the embodiment of the present invention includes a plurality of magnetic nanoparticles 100, a plurality of surfactants 102, coating on the magnetic nanoparticles 100 respectively, and a polar magnetic fluid 110 for delivering the magnetic nanoparticles 100 to a target site.

The magnetic nanoparticles 100 may be Fe₂O₃ magnetic nanoparticles or Fe₃O₄ magnetic nanoparticles, but the invention is not limited thereto. Other possible magnetic nanoparticles, including MnFe₂O₄, CoFe₂O₄, NiFe₂O₄, or Fe₂O₃, may also be utilized and be comprised within the scope of this invention. The diameter of the magnetic nanoparticles 100 is less than 100 nm. Besides, the magnetic nanoparticles 100 are coated by the surfactants 102 respectively. The surfactants 102 may be polar molecules for helping dispersing the magnetic nanoparticles 100 in the polar magnetic fluid 110 or alternatively for improving binding of bio-receptors on tumor cells to the surface of the magnetic nanoparticles 100. Specifically, the surfactant 102 may be dextran. Certainly, other possible surfactant may also be utilized and be comprised within the scope of this invention. Moreover, the polar magnetic fluid 110 for delivering the magnetic nanoparticles 100 coated with the surfactants 102 to a target site may comprise water, phosphate-buffered saline (PBS) solution, or ethanol. Besides, the target site may be referred to tumor cells or tumor tissues.

It should be noted that, the magnetic nanoparticles 100 may also be modified with biomateirals, such as bioprobes which are specific to proteins of tumor cells at the target sites, or chemotherapy drugs for chemotherapy, wherein the bioprobes may include antibodies, DNA, or small peptides or other biomaterials with similar structures. Specifically, the thermal therapeutic reagent 10A as illustrated above may be classified as the following four reagents: thermal therapeutic reagent A, thermal therapeutic reagent C, thermal therapeutic reagent E, and thermal therapeutic reagent G. Thermal therapeutic reagent A includes the polar magnetic fluid containing magnetic nanoparticles that are not modified with bioprobes and chemotherapy drugs. Thermal therapeutic reagent C includes the polar magnetic fluid containing magnetic nanoparticles that are modified with bioprobes but are not modified with chemotherapy drugs. Thermal therapeutic reagent E includes the polar magnetic fluid containing magnetic nanoparticles that are not modified with bioprobes but are modified with chemotherapy drugs. Thermal therapeutic reagent G includes the polar magnetic fluid containing magnetic nanoparticles that are modified with bioprobes and chemotherapy drugs.

Noteworthily, the present embodiment is not limited to use the polar magnetic fluid 110 for delivering the magnetic nanoparticles 100, in another embodiment, a non-polar magnetic fluid may also be used for delivering the magnetic nanoparticles 100 coated with the surfactants 102. Referring to FIG. 1, the thermal therapeutic reagent 10B of the embodiment of the present invention includes a plurality of magnetic nanoparticles 100, a plurality of surfactants 102 coating on the magnetic nanoparticles 100 respectively, and a non-polar magnetic fluid 120 for delivering the magnetic nanoparticles 100 to a target site. Herein, the non-polar magnetic fluid 120 may comprise lipiodol. In addition, the material of magnetic nanoparticles 100, surfactants 102, and other biomaterials modified on the magnetic nanoparticles 100 are similar to those in the previous described embodiment of the thermal therapeutic reagent 10A, so that no further description is made here. The thermal therapeutic reagent 10B as illustrated above may also be classified as the following four reagents: thermal therapeutic reagent B, thermal therapeutic reagent D, thermal therapeutic reagent F, and thermal therapeutic reagent H. Thermal therapeutic reagent B includes the non-polar magnetic fluid containing magnetic nanoparticles that are not modified with bioprobes and chemotherapy drugs. Thermal therapeutic reagent D includes the non-polar magnetic fluid containing magnetic nanoparticles that are modified with bioprobes but are not modified with chemotherapy drugs. Thermal therapeutic reagent F includes the non-polar magnetic fluid containing magnetic nanoparticles that are not modified with bioprobes but are modified with chemotherapy drugs. Thermal therapeutic reagent H includes the non-polar magnetic fluid containing magnetic nanoparticles that are modified with bioprobes and chemotherapy drugs.

It should be noted that, the thermal therapeutic reagents A to H as illustrated above can be concentrated and activated by a magnetic field. The magnetic field may be generated by means of a magnet in the region with the strongest field gradient. For example, the thermal therapeutic reagents A to H may be introduced into the target site and activated by an implantable magnetized device, such as a magnetized needle (e.g., a catheter or puncture needle), a magnetized metal coil or a magnetized embolic material, etc.; the thermal therapeutic reagents A to H may also be activated by a metal probe of radio frequency ablation (RFA) or of microwave ablation, etc.; the thermal therapeutic reagents A to H may also be activated by a an external magnet source, such as a non-implantable magnet or an activated coils. The present embodiment is not limited thereto.

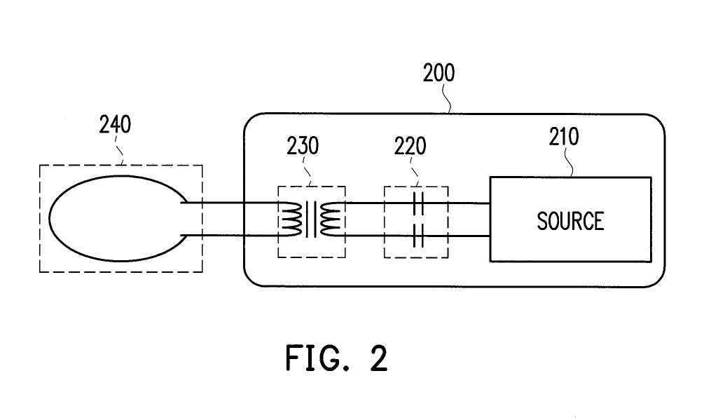

FIG. 2 is a schematic diagram illustrating a magnetic field generator for generating an adjustable magnetic field. Referring to FIG. 2, a magnetic field generator 200 includes a magnetic field source 210, a capacitor 220, a transformer 230, and a coil 240 coupled to the transformer 230, wherein the magnetic field is generated inside the coil 240 and is changed according to the frequency of the magnetic field source 210. Herein, the generated magnetic field may be an oscillating magnetic field, such as an AC magnetic field, an elliptical rotating magnetic field or a rotating magnetic field, etc. Therefore, when thermal therapeutic reagents are located in the target site, the magnetic nanoparticles can be oscillated by the alternating magnetic field, which causes the magnetic nanoparticles to be dispersed within the target site, thus generating heat in the tumor tissue and causing the permanent damage and destruction of tumor cells. In the present application, the coil 240 may be configured on a puncture needle or a magnetized embolic material which deliver the thermal therapeutic reagents A to H. The coil 240 may also be a metal probe of radio frequency ablation (RFA) or of microwave ablation. In addition, the coil 240 may serve as a non-implantable magnet or an activated coil that surround the target site. As such, the thermal therapeutic reagents A to H can be activated under the alternating magnetic field at the target site.

In the following below, treatment methods using the thermal therapeutic reagents A to H are described.

Using the Thermal Therapeutic Reagent to Increase Healing Efficacy of Thermal Ablative Therapies in Locoregional Treatment.

Radio-frequency ablation (RFA) or microwave ablation (MWA) are procedures used to treat a variety of inoperable tumors. RFA/MWA may use energy delivered through a metal probe inserted into a tumor tissue under radiographic guidance. Since, the mechanisms of RFA or MCT are based on the generation of heat between the tumor tissue and electric current or microwave emitted by an RF or microwave electrode into the tumor, the heat generated in the tumor tissue treated with RFA/MWA energy may cause permanent damage and destruction of tumor cells. Herein, the thermal therapeutic reagent A, including the polar magnetic fluid and contains magnetic nanoparticles that are not modified with bioprobes and chemotherapy drugs, may be applied in the treatment of RFA/MWA. Specifically, thermal therapeutic reagent A may be encapsulated in a metal carrier and being injected at the local region of the tumor tissues. With magnets, the magnetic nanoparticles could aid heat dissipation since the heat transfer coefficient is higher. Therefore, thermal therapeutic reagent A may enhance heat transfer of the heat generated with RFA/MWA energy at the local region. As such, the fast-delivered energy may cause atoms in tumor cells to vibrate and create friction, which leads to death of tumor cells.

Using the Thermal Therapeutic Reagent to Increase Specificity of Thermal Ablative Therapies in Locoregional Treatment.

Thermal therapeutic reagent A, including the polar magnetic fluid and contains magnetic nanoparticles that are not modified with bioprobes and chemotherapy drugs, may also be applied and introduced into the tumor by way of an implantable magnetic needle. Besides, the implantable magnetic needle may attract the magnetic nanoparticles which may further congregate around the target site. Similarly, under the magnetic field, thermal therapeutic reagent A may be activated, and thus due to the congregation of the magnetic nanoparticles, the heat generated by the activated magnetic nanoparticles in the thermal therapeutic reagent A may be more intensive. Since thermal therapeutic reagent A could aid heat dissipation with higher heat transfer coefficient, the fast-delivered energy may cause atoms in tumor cells to vibrate and create friction, which leads to death of tumor cells.

Thermal therapeutic reagent B, including the non-polar magnetic fluid containing magnetic nanoparticles that are not modified with bioprobes and chemotherapy drugs, may be applied in the treatment of transcatheter arterial embolizaton. The treatment of combining the use of the thermal therapeutic reagent and transcatheter arterial embolizaton is called transcatheter arterial hyperthermic embolization (TAHE) herein. Specifically, lipiodol, for example, may be used as an embolic material used in transcatheter arterial embolizaton. Since lipiodol may be selectively accumulated and stays longer in the neovessels of liver tumor tissues, lipiodol is able to deliver magnetic nanoparticles in thermal therapeutic reagent B to tumor tissues. Therefore, when the magnetic field is applied as the magnetic nanoparticles are delivered to tumor tissues, the magnetic nanoparticles may be activated and generate intensive heat that cause atoms in tumor cells to vibrate and create friction, which leads to death of tumor cells.

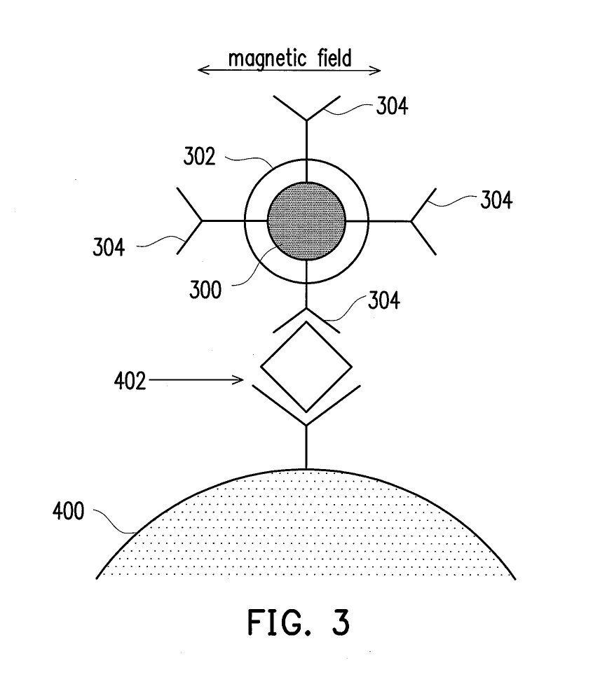

It should be noted that, tumor antigens produced in tumor cells are useful tumor markers in identifying tumor cells. Specifically, the magnetic nanoparticles modified with bioprobes will be targeted at a preferentially expressed protein in the tumour cells by binding to tumor antigens through the bioprobes. Therefore, these bioprobe-targeted magnetic nanoparticles may greatly reduce side effects in the treatment of cancer. FIG. 3 is a schematic diagram illustrating a thermal therapeutic reagent binding on a tumor cell. Referring to FIG. 3, the magnetic nanoparticles 300 coated with surfactant 302 and modified with bioprobes 304 binds to a tumor antigen 402 of a tumor cell 400 through the bioprobes 304.

Herein, thermal therapeutic reagent C, including the polar magnetic fluid containing magnetic nanoparticles that are modified with bioprobes but are not modified with chemotherapy drugs, and thermal therapeutic reagent D, including the non-polar magnetic fluid containing magnetic nanoparticles that are modified with bioprobes but are not modified with chemotherapy drugs, may be delivered to the target site by binding to the tumor cells through the bioprobes. In other words, the bioprobes modified on the magnetic nanoparticles may serve as molecular targeted agents to bind to the tumor cells. Therefore, thermal therapeutic reagent C and thermal therapeutic reagent D may be delivered to the target site by being injected into veins (or at the target site) without using an implantable needle or other embolic materials. Accordingly, the magnetic nanoparticles may generate intensive heat when activated by the magnetic field, which leads to death of tumor cells.

Using the Thermal Therapeutic Reagent to Increase Specificity of Thermal Ablative Therapies Combining Chemotherapy.

Chemotherapy drugs or anticancer drugs may be combined in the thermal therapeutic reagent to enhance the healing efficacy. In one embodiment, thermal therapeutic reagent E, including the polar magnetic fluid containing magnetic nanoparticles that are not modified with bioprobes but are modified with chemotherapy drugs, may be applied and introduced into the tumor by way of an implantable magnetic needle. Sine thermal therapeutic reagents E could aid heat dissipation with higher heat transfer coefficient, the fast-delivered energy may cause atoms in tumor cells to vibrate and create friction. Similarly, the heat generated by the activated magnetic nanoparticles in the thermal therapeutic reagent E may be more intensive due to the congregation of the magnetic nanoparticles by the implantable magnetic needle. Moreover, in addition to the heat generated by the magnetic nanoparticles when activated, the chemotherapy drugs modified on the magnetic nanoparticles may be released to tumor tissues at the same time. Accordingly, the healing efficacy to cause the death of tumor cells will be enhanced.

In one embodiment, thermal therapeutic reagent F, including the non-polar magnetic fluid containing magnetic nanoparticles that are not modified with bioprobes but are modified with chemotherapy drugs, may be applied in the treatment of transcatheter arterial embolizaton in which lipiodol may be used as an embolic material in transcatheter arterial embolizaton. Herein, in addition to the heat generated by the magnetic nanoparticles when activated, the chemotherapy drugs modified on the magnetic nanoparticles may be released to tumor tissues, which is able to enhance the effect to cause the death of tumor cells.

It should also be noted that, in addition to being modified with chemotherapy drugs, the thermal therapeutic reagent may also be modified with bioprobes specific to proteins on tumor cells so as to be targeted at the tumor tissue by binding to the tumor cells, which will further enhance the healing efficacy.

In one embodiment, thermal therapeutic reagent G, including the polar magnetic fluid containing magnetic nanoparticles that are modified with bioprobes and chemotherapy drugs, and thermal therapeutic reagent H, including the non-polar magnetic fluid containing magnetic nanoparticles that are modified with bioprobes and chemotherapy drugs, may be delivered to the target site by binding to the tumor cells through the modified bioprobes. Herein, the treatment of combining the use of the thermal therapeutic reagent, bioprobes and chemotherapy drugs is called concurrent chemohyperthermic therapy (CCHT). Similar to thermal therapeutic reagent C and D, thermal therapeutic reagent G and H may also be delivered to the target site by being injected into veins (or at the target site) without using an implantable needle or other embolic materials. In addition to the heat generated by the magnetic nanoparticles when activated, the chemotherapy drugs modified on the magnetic nanoparticles may be released to tumor tissues at the same time. As such, with the use of molecular-targeted therapy and chemotherapy, thermal therapeutic reagent G and H may further help enhance the healing efficacy to cause the death of tumor cells.

In summary, in the provided thermal therapeutic reagents, the magnetic nanoparticles may enhance heat transfer of the heat generated around tumor tissues during locoregional treatments. Based on this mechanism, the magnetic nanoparticles may also be modified with biomateirals, such as bioprobes which are specific to proteins of tumor cells at the target sites, or chemotherapy drugs for chemotherapy. In other words, the bioprobes modified on the magnetic nanoparticles may serve as molecular targeted agents to bind to the tumor cells, and thus the use of the thermal therapeutic reagent will greatly reduce side effects in the treatment of cancer. With the modified chemotherapy drugs, the chemotherapy drugs may also be released to tumor cells, thus interfering with the tumor cells' ability to grow or reproduce. Accordingly, with the use of the provided thermal therapeutic reagents, the healing efficacy to cause the death of tumor cells will be improved.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A thermal therapeutic reagent comprising:
a plurality of magnetic nanoparticles;
a plurality of surfactants, coating on the magnetic nanoparticles respectively; and
a polar magnetic fluid for delivering the magnetic nanoparticles to a target site,
wherein the magnetic nanoparticles are capable of being activated under a magnetic field applied at the target site.

2. The thermal therapeutic reagent as claimed in claim 1, wherein the magnetic nanoparticles are Fe₂O₃ magnetic nanoparticles or Fe₃O₄ magnetic nanoparticles.

3. The thermal therapeutic reagent as claimed in claim 1, wherein the diameter of the magnetic nanoparticles is less than 100 nm.

4. The thermal therapeutic reagent as claimed in claim 1, wherein the surfactant is dextran.

5. The thermal therapeutic reagent as claimed in claim 1, wherein the thermal therapeutic reagent is injected at the target site through a magnetic needle.

6. The thermal therapeutic reagent as claimed in claim 1, wherein the thermal therapeutic reagent is applied to microwave ablation, or radiofrequency ablation.

7. The thermal therapeutic reagent according to claim 1, wherein the magnetic nanoparticles are further modified with bioprobes which are specific to proteins of tumor cells at the target sites.

8. The thermal therapeutic reagent as claimed in claim 1, wherein the magnetic nanoparticles are further modified with chemotherapy drugs.

9. The thermal therapeutic reagent as claimed in claim 1, wherein the magnetic nanoparticles are further modified with bioprobes and chemotherapy drugs.

10. The thermal therapeutic reagent as claimed in claim 1, wherein the polar magnetic fluid comprises water, phosphate-buffered saline (PBS) solution, or ethanol.

11. A thermal therapeutic reagent comprising:
a plurality of magnetic nanoparticles;
a plurality of surfactants, coating on the magnetic nanoparticles respectively; and
a non-polar magnetic fluid containing an embolic material for delivering the magnetic nanoparticles to a target site,
wherein the magnetic nanoparticles are capable of being activated under the a magnetic field applied at the target site.

12. The thermal therapeutic reagent as claimed in claim 11, wherein the magnetic nanoparticles are Fe₂O₃ magnetic nanoparticles or Fe₃O₄ magnetic nanoparticles.

13. The thermal therapeutic reagent according to claim 11, wherein the diameter of the magnetic nanoparticles is less than 100 nm.

14. The thermal therapeutic reagent as claimed in claim 11, wherein the surfactant is dextran.

15. The thermal therapeutic reagent as claimed in claim 11, wherein the thermal therapeutic reagent is applied in transcatheter arterial embolization.

16. The thermal therapeutic reagent as claimed in claim 11, wherein the thermal therapeutic reagent is applied to microwave ablation, or radiofrequency ablation.

17. The thermal therapeutic reagent as claimed in claim 11, wherein the magnetic nanoparticles are further modified with bioprobes which are specific to proteins of tumor cells at the target sites.

18. The thermal therapeutic reagent as claimed in claim 11, wherein the magnetic nanoparticles are further modified with chemotherapy drugs.

19. The thermal therapeutic reagent as claimed in claim 11, wherein the magnetic nanoparticles are further modified with bioprobes and chemotherapy drugs.

20. The thermal therapeutic reagent as claimed in claim 11, wherein the non-polar magnetic fluid comprises lipiodol.
